(19) United States
(12) Patent Application Publication
(10) Pub. No.: US 2026/0338803 A1
(43) Pub. Date: Oct. 29, 2026

(54) A DETECTION METHOD

(71) Applicant: Akershus Universitetssykehus HF, Lørenskog (NO)

(72) Inventor: Tormod FLADBY, Lørenskog (NO)

(73) Assignee: Akershus Universitetssykehus HF, Lørenskog (NO)

(21) Appl. No.: 19/109,894

(22) PCT Filed: Aug. 29, 2023
(86) PCT No.: PCT/EP2023/073607

§ 371 Date: Mar. 7, 2025

(30) Foreign Application Priority Data
Sep. 9, 2022 (EP) 22194982.9

(51) Int. Cl. G01N 33/68

(52) U.S. Cl. CPC G01N 33/6896; G01N 2333/4709; G01N 2800/2871

(57) ABSTRACT

A method of detecting, predicting or monitoring the progress of amyloid angiopathy, ARIA (amyloid-related imaging abnormalities) or CAA (cerebral amyloid angiopathy) in an individual. The method comprises the step of: determining the presence or quantity of an Aβ protein fragment in a biological sample obtained from the individual, wherein the Aβ protein fragment is an Aβ₁₋₄₀ or Aβₓ₋₃₄ fragment or a fragment of Aβ₁₋₄₀.

### FIELD OF THE INVENTION

The present invention relates to a method of detecting, predicting or monitoring the progress of amyloid angiopathy, ARIA (amyloid-related imaging abnormalities) or CAA (cerebral amyloid angiopathy) in an individual. The present invention also relates to a method of detecting an Aβ protein fragment.

### BACKGROUND

Alzheimer's Disease (AD) is an irreversible, progressive brain disorder that slowly destroys memory and thinking skills, and eventually, the ability to carry out even simple tasks. While the specific causes of AD are not fully known, it is characterized by changes in the brain—including amyloid-β (Aβ) plaques and neurofibrillary, or tau, tangles—that result in loss of neurons and their connections. Currently the leading hypothesis—the “amyloid cascade”—proposes that accumulation of Aβ resulting from an imbalance between Aβ production and Aβ clearance in the brain is the driving force causing the disease. In particular, Aβ clearance involves clearance to CSF and to blood through brain small vessel walls, and degradation to shorter fragments. Microglia (resident brain innate immune cells) are main players in Aβ parenchymal degradation, while perivascular macrophages and astrocytes further contribute to clearance. Pericytes lining the brain small vessel endothelium usually release “detoxified” Aβ34 to the CSF, but, in AD, pericytes may develop impaired Aβ clearance capacity (Henjum, K. et al. “Analyzing microglial-associated Abeta in Alzheimer's disease transgenic mice with a novel mid-domain Abeta-antibody”, 2020, Sci Rep 10, 10590). Thus, it is hypothesized that removal of Aβ plaques will reduce cognitive decline.

One approach that has garnered a significant amount of attention and resources for its potential as a disease modifying approach is passive immunotherapy; Aβ-directed monoclonal antibodies—directed at clearing/removing Aβ. Several new drugs such as Aducanumab (trade name Aduhelm) (Biogen), Gantenerumab (Roche), Donanemab (Lilly), and Lecanemab (Eisai/BioArctic) are expected to reach the market in the near future. Aduhelm is the first new AD treatment to be launched in over 20 years, as well as being both the first approved immunotherapy and the first therapy that targets the fundamental pathophysiology of the disease. The clinical trials of Aduhelm demonstrated a statistically significant cognitive improvement in patients with prodromal or mild AD, together with a dose- and time-dependent reduction of deposited Aβ on amyloid-Positron Emission Tomography (PET) scan. Unfortunately, and similarly to previously reported results with different Aβ-directed monoclonal antibodies, roughly 40% of Alzheimer's patients taking Aduhelm experienced a side effect called Amyloid-Related Imaging Abnormalities, or ARIA, a serious swelling of the brain, in worst cases leading to bleeding, lasting brain damage and death. It is thought that ARIA occurs as a result of a chain of events. Aβ antibodies cause the release of Aβ from neuritic plaques, and if the patient's Aβ clearance capacity is low, Aβ accumulation destroys the blood brain barrier (BBB) and the vessel walls will leak resulting in swelling and eventually bleeding. In other words, ARIA is understood to be caused by defective CNS pericytes causing vascular leakage through a defective blood brain barrier. Of note, ARIA is reported to be more represented in patients treated at the higher, and more effective, dosages of the administered treatment.

In addition, small vessel disease (SVD), which includes amyloid angiopathy (AA) and cerebral amyloid angiopathy (CAA), increases the risk of AD. Impaired Aβ clearance is linked to amyloid plaque formation, as mentioned above, and cerebral amyloid angiopathy (CAA). Incipient CAA putatively increases susceptibility for ARIA in AD patients.

These facts dramatically increase the need for finding biomarkers/IVD tests for predicting, diagnosing and monitoring these hazardous side effects, i.e. ARIA.

Other pathologies for which there is currently no biomarker, and which it is not currently possible to diagnose in vivo, are amyloid angiopathy and cerebral amyloid angiopathy (CAA). In addition, evidence is pointing to amyloid angiopathy being a feature of early AD. Early detection as a prerequisite for the development of treatments of incipient amyloid angiopathy in AD will also be valuable.

The present invention seeks to alleviate the above problems.

### SUMMARY OF THE INVENTION

The present invention arises from a recognition that Alzheimer's Disease (AD), amyloid angiopathy, cerebral amyloid angiopathy (CAA) and amyloid-related imaging abnormalities (ARIA) may be a quartet with the Aβ protein as a common denominator. A substantial body of literature is pointing towards a direct link between neurodegenerative and cerebrovascular pathologies, where AD and CAA stand out as clear examples. Each of their underlying pathogenic pathways intersect at the level of Aβ generation and its clearance from the brain. CAA involves Aβ deposition in small vessel walls (mainly Aβ₁₋₄₀), whereas Aβ deposits in AD are also found in neuritic plaques (mainly Aβ₁₋₄₂). Furthermore, several pathways are known to be involved in clearing Aβ from the brain, including transport across the blood-brain barrier, phagocytosis, enzymatic degradation and perivascular drainage (through two mechanisms intramural periarterial drainage to the blood stream and the perivascular, via the glymphatic system, CSF efflux). The extent to which each of these pathways contribute to elimination of Aβ from the brain is unclear, but abnormal perivascular drainage of Aβ is commonly believed to play a major role in the pathogenesis of CAA, AD and ARIA, and to be reflected in amyloid angiopathy. Thereby, ARIA is yet another intersection between amyloid angiopathy, CAA and AD, representing an overload on the perivascular clearance pathways. The effects of removing Aβ from amyloid plaques appear to cause a vicious cycle by aggravating the Aβ clearance system.

Furthermore, the present invention arises from the observation that, in a study of AD patients, the reduced presence of Aβ₁₋₄₀ and Aβₓ₋₃₄ fragments in CSF samples obtained from the patients correlated with an increase of the PSMD (peak skeletonized mean diffusivity) measure determined in the patients. In particular, it is accepted that PSMD is a robust imaging marker for SVD (e.g. amyloid angiopathy and CAA; Baykara et al. “A novel imaging marker for Small Vessel Disease based on skeletonization of white matter tracts and diffusion histograms”, Ann Neurol, 2016 October, 80(4), p. 581-92; Horn et al. “Peak width of skeletonized mean diffusivity and cognitive performance in cerebral amyloid angiopathy”, Front. Neurosci., 2023, vol. 17), such that the PSMD measure is indicative of the presence of or propensity for amyloid angiopathy, CAA (cerebral amyloid angiopathy) and/or ARIA (amyloid-related imaging abnormalities) in individuals. Moreover, it has been shown that Aβ₁₋₄₀ and Aβₓ₋₃₄ fragments indicate the presence of brain small vessel amyloid angiopathy.

As pericytes, at blood vessel walls, are the site of degradation of Aβ, particularly to the Aβₓ₋₃₄ fragment, and pericytes have close connections to both the glymphatic system and blood vessel capillary lumens, it is expected that blood plasma reflects CSF levels of Aβ1-40, fragments of Aβ₁₋₄₀ and Aβₓ₋₃₄.

In a first aspect, the present invention provides a method of detecting, predicting or monitoring the progress of amyloid angiopathy, ARIA (amyloid-related imaging abnormalities) or CAA (cerebral amyloid angiopathy) in an individual comprising the step of determining the presence or quantity of an Aβ protein fragment in a biological sample obtained from the individual, wherein the Aβ protein fragment is an Aβ₁₋₄₀ or Aβₓ₋₃₄ fragment. In some embodiments, the Aβ protein fragment is a fragment of Aβ₁₋₄₀ or is Aβₓ₋₃₄ (i.e. an Aβₓ₋₃₄ fragment).

Preferably, a decrease in the quantity of the Aβ protein fragment in the sample is indicative of the presence, predicted presence or development of the progress of amyloid angiopathy, ARIA or CAA in the individual, preferably wherein the decrease is with respect to the quantity of the Aβ protein fragment in a biological sample previously obtained from the individual or with respect to the quantity of the Aβ protein fragment in a biological sample obtained from a healthy individual or with respect to the quantity of the Aβ protein fragment in biological samples obtained from a plurality of healthy individuals.

In a second aspect, the present invention provides a method of detecting an Aβ protein fragment, comprising (i) obtaining a biological sample from an individual having or suspected of having amyloid angiopathy, ARIA (amyloid-related imaging abnormalities) or CAA (cerebral amyloid angiopathy) and (ii) detecting amyloid angiopathy, ARIA (amyloid-related imaging abnormalities) or CAA (cerebral amyloid angiopathy) by detecting the Aβ protein fragment in the biological sample, wherein the Aβ protein fragment is an Aβ₁₋₄₀ or Aβₓ₋₃₄ fragment. Preferably, the Aβ protein fragment is the Aβₓ₋₃₄ fragment or a fragment of Aβ₁₋₄₀.

The following statements apply to both the first and second aspects of the invention.

Advantageously, the biological sample is a CSF sample or a blood sample, preferably a blood sample.

Conveniently, the Aβ₁₋₄₀ fragment, or the fragment of Aβ₁₋₄₀, has the amino acid sequence of SEQ ID NO: 2 and the Aβₓ₋₃₄ fragment consists of a fragment of SEQ ID NO:2 having at its N-terminal an amino acid at a position of amino acid residue 21 or lower and at its C-terminal the amino acid at amino acid residue 34.

Preferably the Aβₓ₋₃₄ fragment comprises or consists of the amino acid sequence of SEQ ID NO: 6.

Advantageously, determining or detecting the Aβ₁₋₄₀ or Aβₓ₋₃₄ fragment, or the fragment of Aβ₁₋₄₀, comprises detecting a region of Aβ₁₋₄₀ comprising at least 4 amino acids including residue 21, residue 34 or residue 40 of Aβ₁₋₄₀.

Conveniently, determining or detecting the Aβ₁₋₄₀ or Aβₓ₋₃₄ fragment, or the fragment of Aβ₁₋₄₀, comprises detecting a region of the fragment comprising at least 4 amino acids comprising residues 21-24, residues 31-34 or residues 39-40 of Aβ₁₋₄₀ (SEQ ID NO: 2).

Advantageously, determining or detecting the Aβ₁₋₄₀ or Aβₓ₋₃₄ fragment, or the fragment of Aβ₁₋₄₀, comprises detecting the N-terminal of SEQ ID NO: 6, the C-terminal of SEQ ID NO: 6, a region of the fragment comprising residues 21-29 of Aβ₁₋₄₀ and/or a region of the fragment comprising residues 34-40 of Aβ₁₋₄₀ (SEQ ID NO: 2).

Conveniently, determining or detecting the Aβ₁₋₄₀ or Aβₓ₋₃₄ fragment, or the fragment of Aβ₁₋₄₀, comprises detecting more than one of the N-terminal or C-terminal of SEQ ID NO: 6 and the regions of Aβ₁₋₄₀, preferably by detecting
i) a region of the fragment comprising at least 4 amino acids comprising residues 21-24 and a region of the fragment comprising at least 4 amino acids comprising residues 31-34 or 39-40,
ii) the N-terminal of SEQ ID NO: 6 and a region of the fragment comprising residues 34-40 of or Aβ₁₋₄₀, preferably residues 1-6 of SEQ ID NO: 6 and a region of the fragment comprising residues 34-40 of Aβ₁₋₄₀;
iii) the C-terminal of SEQ ID NO: 6 and a region of the fragment comprising residues 21-24, preferably 21-29, of or Aβ₁₋₄₀; and/or
iv) a region of the fragment comprising residues 21-24, preferably residues 21-29, of Aβ₁₋₄₀ and a region of the fragment comprising residues 34-40 of Aβ₁₋₄₀.

Preferably, the Aβ protein fragment is a fragment which has cleaved between amino acid residues 19 and 20 and/or between amino acid residues 34 and 35. The Aβ protein fragment may be a fragment of Aβ₁₋₄₀ or the Aβₓ₋₃₄ fragment.

Conveniently, determining or detecting the Aβ₁₋₄₀ or Aβₓ₋₃₄ fragment, or the fragment of Aβ₁₋₄₀, comprises using antibodies to detect the Aβ₁₋₄₀ or Aβₓ₋₃₄ fragment, or the fragment of Aβ₁₋₄₀. Preferably, an antibody specific for SEQ ID NO: 3 or 7 and an antibody specific for the C-terminal of SEQ ID NO: 6 or specific for SEQ ID NO: 4 are used. Advantageously, an antibody specific for SEQ ID NO: 5, 8 or 9 can also be used.

Advantageously, the method comprises determining the quantity of the Aβₓ₋₃₄ fragment and the quantity of Aβ₁₋₄₀ (SEQ ID NO: 2) in the sample, and calculating a ratio thereof.

Conveniently, a decrease in the quantity of the Aβₓ₋₃₄ fragment relative to the quantity of Aβ₁₋₄₀ is indicative of the presence, predicted presence or development of the progress of amyloid angiopathy, ARIA or CAA in the individual, preferably wherein the decrease is with respect to the ratio of the quantity of the Aβₓ₋₃₄ fragment to Aβ₁₋₄₀ calculated from a biological sample previously obtained from the individual or with respect to the ratio of the quantity of the Aβₓ₋₃₄ fragment to Aβ₁₋₄₀ calculated from a biological sample obtained from a healthy individual or with respect to the ratio of the quantity of the Aβₓ₋₃₄ fragment to Aβ₁₋₄₀ calculated from biological samples obtained from a plurality of healthy individuals.

### BRIEF DESCRIPTION OF THE FIGURES

FIG. 1 is a scatterplot of the presence of the Aβₓ₋₃₄ fragment in AD patients also measured for the PSMD (peak skeletonized mean diffusivity) measure.

FIG. 2 is a scatterplot of the presence of the Aβ₁₋₄₀ fragment in AD patients also measured for the PSMD (peak skeletonized mean diffusivity) measure.

FIG. 3 is scatterplots of the relationships between the Aβₓ₋₃₄ and Aβ₁₋₄₀ fragments in CSF and PSMD, by brain lobe and in total. A−/T−/N−: subjects with negative markers for amyloid plaques, tau-tangles and neurodegeneration, representative of healthy subjects; A+/T−/N−: subjects with positive markers for amyloid plaques, and negative markers for tau-tangles and neurodegeneration, representative of subjects with early AD; A+/T or N+: subjects with positive markers for amyloid plaques and either, or both, positive markers for tau-tangles and neurodegeneration, representative of subjects with advanced AD.

### BRIEF DESCRIPTION OF THE SEQUENCE LISTING

SEQ ID NO: 1 is the amino acid sequence of Aβ₁₋₄₂.

SEQ ID NO: 2 is the amino acid sequence of Aβ₁₋₄₀.

SEQ ID NO: 3 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 4 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 5 is the amino acid sequence of an epitope of Aβ₁₋₄₂ (SEQ ID NO: 1) against which an antibody was raised.

SEQ ID NO: 6 is the amino acid sequence of a mid-domain fragment of Aβ₁₋₄₂ and Aβ₁₋₄₀, namely, residues 20-34 of Aβ₁₋₄₂ (SEQ ID NO: 1) and Aβ₁₋₄₀ (SEQ ID NO: 2).

SEQ ID NO: 7 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 8 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 9 is the amino acid sequence of an epitope of Aβ₁₋₄₂ (SEQ ID NO: 1) and A_{β1-40} (SEQ ID NO: 2) for detecting a mid-domain fragment of Aβ₁₋₂ (SEQ ID NO: 1) and Aβ₁₋₄₀ (SEQ ID NO: 2).

### DETAILED DESCRIPTION

In general terms, the present invention concerns a method of detecting, predicting or monitoring the progress of (hereinafter abbreviated to “detecting”) amyloid angiopathy, ARIA or CAA in an individual. The individual may, for example, be undergoing, or may be about to undergo, an anti-amyloid treatment, such as a treatment for AD (such as passive immunotherapy with an anti-amyloid drug such as Aducanumab, Gantenerumab, Donanemab, or Lecanemab) which may give rise to an increased propensity for ARIA.

The present invention concerns a method which comprises determining the presence or quantity of an Aβ protein fragment in a biological sample (e.g. blood or CSF) obtained from the individual. The rationale for this aspect of the invention is that Aβ protein fragment (e.g. Aβₓ₋₃₄) measurement reflects the ongoing CNS Aβ clearance of Aβ₁₋₄₂ and Aβ₁₋₄₀, taking place in brain glia and MPS cells (i.e. microglia, perivascular macrophages and pericytes). Defective clearance releases less Aβ₁₋₄₀ to Aβₓ₋₃₄, e.g. Aβ₁₋₃₄ or Aβ₂₀₋₃₄, which can thereby be measured in, for example, CSF or plasma.

In some embodiments, the individual is suspected of having amyloid angiopathy, ARIA or CAA.

Determining Aβ Protein Fragment in a Biological Sample Obtained from an Individual

In general terms, this concept relates to method of detecting, predicting or monitoring the progress of amyloid angiopathy, ARIA (amyloid-related imaging abnormalities) or CAA (cerebral amyloid angiopathy) in an individual comprising the step of determining the presence or quantity of an Aβ protein fragment in a biological sample obtained from the individual, wherein the Aβ protein fragment is an Aβ₁₋₄₀ or Aβₓ₋₃₄ fragment or a fragment of A₁₋₄₀.

In some embodiments, a decrease in the quantity of the Aβ protein fragment in the sample is indicative of the presence, predicted presence or development of the progress of amyloid angiopathy, ARIA or CAA in the individual. For example, in some embodiments, a biological sample is obtained from the individual at more than one time point and the quantity of the Aβ protein fragment in the biological sample at each time point is compared such that a decrease over time is indicative of the presence, predicted presence or development of the progress of amyloid angiopathy, ARIA or CAA in the individual. In alternative embodiments, a comparison is instead made with the quantity of Aβ protein fragment in a biological sample obtained from a healthy individual (or in an average or standard derived from biological samples obtained from multiple healthy individuals), whereby a decrease in the quantity of the Aβ protein fragment in the individual as compared with the healthy individual or individuals is indicative of the presence, predicted presence or development of the progress of amyloid angiopathy, ARIA or CAA in the individual.

In some embodiments, the presence or quantity of the Aβ protein fragment is determined in more than one biological sample obtained from an individual at different time points, for example at two, three, four or five time points. The time points may each be, for example, one, two, three, four, five or six months apart, or may each be one, two or three years apart. The presence or quantity of the Aβ protein fragment in each biological sample may be compared across the time points, and a decrease in the quantity of the Aβ protein fragment across the time points is indicative of the presence, predicted presence, development of the progress of amyloid angiopathy, ARIA or CAA.

The invention also relates to a method of detecting an Aβ protein fragment, comprising (i) obtaining a biological sample from an individual having or suspected of having amyloid angiopathy, ARIA (amyloid-related imaging abnormalities) or CAA (cerebral amyloid angiopathy) and (ii) detecting amyloid angiopathy, ARIA (amyloid-related imaging abnormalities) or CAA (cerebral amyloid angiopathy) by detecting the Aβ protein fragment in the biological sample, wherein the Aβ protein fragment is an Aβ₁₋₄₀ or Aβₓ₋₃₄ fragment, preferably the Aβₓ₋₃₄ fragment or a fragment of Aβ₁₋₄₀. In preferred embodiments, the individual is suspected of having amyloid angiopathy, ARIA or CAA.

The biological sample may be a CSF sample or a blood sample. When the biological sample is a blood sample, it may be a plasma sample. In some embodiments, the biological sample is a CSF sample.

In some embodiments, the method comprises the step of obtaining the biological sample from the individual before determining the presence or quantity of an Aβ protein fragment in the biological sample.

Detection of the Aβ Protein Fragment and/or at Least One Fragment Thereof

The step of detecting the amount of the Aβ protein fragment and/or the at least one fragment thereof in the samples can be carried out using any techniques known in the art. For example, the step of detecting the Aβ protein fragment and/or the at least one fragment thereof may be using an immunoassay, Mass Spectrometry techniques, semi-quantitative techniques such as Western Blot, microscopy, flow cytometry, or ELISA techniques.

The step of detecting the Aβ protein fragment and/or at least one fragment thereof can detect any part of the Aβ protein fragment and/or at least one fragment thereof. In some embodiments, the detection comprises detecting one end of the Aβ protein fragment and/or at least one fragment thereof, for example, the N-terminal or the C-terminal thereof.

In some embodiments, the method comprises detecting two or more fragments of the Aβ protein fragment Preferably, the method comprises detecting two, and more preferably three, fragments of the Aβ protein fragment.

The step of detecting the Aβ protein fragment and/or at least one fragment thereof may use any suitable detection technique known in the art. In some embodiments, the step of detecting the Aβ protein fragment and/or at least one fragment thereof uses antibody detection.

### AP Protein Fragment and Fragment Thereof

The Aβ protein fragment is an Aβ₁₋₄₀ or Aβₓ₋₃₄ fragment or a fragment of Aβ₁₋₄₀. Aβ₁₋₄₀ preferably has the amino acid sequence of SEQ ID NO: 2. Shorter fragments of Aβ₁₋₄₀ also exist due to cleavage, for example, between residues 18 and 19, residues 19 and 20, residues 20 and 21, residues 33 and 34 and/or residues 34 and 35 (Rogeberg et al., 2015; Henjum et al., 2020), such that it is possible to detect catalysis products by detection of the N- and/or C-terminals of these mid-domain fragments.

Thus, in some embodiments, the method comprises detecting at least one fragment of Aβ₁₋₄₀ (SEQ ID NO: 2). In some embodiments, the method comprises detecting more than one fragment of Aβ₁₋₄₀ (SEQ ID NO: 2). In some embodiments, the method comprises detecting two, and preferably three, fragments of Aβ₁₋₄₀ (SEQ ID NO: 2).

The method can comprise detecting at least one fragment of Aβ₁₋₄₀ (SEQ ID NO: 2) which is Aβ₁₋₄₀ (SEQ ID NO: 2) which has been cleaved between amino acid residues 19 and 20 thereof and/or between amino acid residues 34 and 35 thereof.

In some embodiments, the at least one fragment is Aβ₁₋₄₀ (SEQ ID NO: 2) which has been cleaved between amino acid residues 19 and 20 thereof. These fragments are referred to herein as “Aβ₂₀₋ₓ” or the “Aβ₂₀₋ₓ fragment”. In these embodiments, the at least one fragment may be detected by detecting a region of the fragment which comprises at least 4 amino acids including residue 21 of Aβ₁₋₄₀ (SEQ ID NO: 2). The at least one fragment may be detected by detecting a region of the fragment comprising residues 21-24, preferably by detecting residues 21-29, of Aβ₁₋₄₀ (SEQ ID NO: 2). In some embodiments, the at least one fragment may be detected by detecting the N-terminal of SEQ ID NO: 6. In some embodiments, the at least one fragment is detected by detecting any one of the aforementioned regions comprising residue 21 or detecting the N-terminal of SEQ ID NO: 6 and, in addition, detecting a region of the fragment which comprises at least 4 amino acid residues including residue 34 or residue 40 of Aβ₁₋₄₀ (SEQ ID NO: 2), preferably a region of the fragment comprising residues 34-40 of Aβ₁₋₄₀ (SEQ ID NO: 2), and more preferably detecting SEQ ID NO: 9. In some embodiments, the at least one fragment is detected by detecting a region of the fragment comprising residues 21-24 of Aβ₁₋₄₀ (SEQ ID NO: 2) and detecting a region of the fragment comprising residues 34-40 of Aβ₁₋₄₀ (SEQ ID NO: 2). In some embodiments, the at least one fragment is detected by detecting the N-terminal of SEQ ID NO: 3 and a region of the fragment comprising residues 34-40 of Aβ₁₋₄₀ (SEQ ID NO: 2), preferably by detecting the N-terminal of SEQ ID NO: 6 and detecting SEQ ID NO: 9. In some embodiments, the at least one fragment is detected using an antibody which is specific to the N-terminal of SEQ ID NO: 6, preferably using an antibody is specific to SEQ ID NO: 3. In some embodiments, the at least one fragment is detected using an antibody specific to the region of the fragment comprising residues 34-40 of Aβ₁₋₄₀ (SEQ ID NO: 2), preferably using an antibody specific to SEQ ID NO: 9. In some embodiments, the at least one fragment is detected using an antibody specific to the N-terminal of SEQ ID NO: 6 and an antibody specific to SEQ ID NO: 9.

In some embodiments, the at least one fragment is Aβ₁₋₄₀ (SEQ ID NO: 2) which has been cleaved between amino acid residues 34 and 35 thereof (i.e. its C-terminal is the amino acid residue at position 34) and whose N-terminal is the amino acid residue at position 21 or an amino acid at a lower position (i.e. position 20, 19, 18 etc. but higher than position 1). These fragments are referred to herein as “Aβₓ₋₃₄” or the “Aβₓ₋₃₄ fragment”. In particular, fragments of Aβ which have been cleaved between amino acid residues 34 and 35 can be detected using antibodies which are specific for this cut site, but which do not distinguish between N-terminal amino acids, as used for example in Examples 1 and 2. This means that an increased number of Aβₓ₋₃₄ fragments (which reflect MPS cell functionality and viability, as discussed above) can be detected, providing a more accurate representation of MPS function and Aβ clearance. In some embodiments, the Aβx-34 fragment is a fragment which has been cleaved between residues 34 and 35 of Aβ₁₋₄₀ and between residues 14 and 15, residues 15 and 16, residues 16 and 17, residues 17 and 18, residues 18 and 19 or residues 19 and 20 of Aβ₁₋₄₀. In embodiments where the fragment is the Aβₓ₋₃₄ fragment, the at least one fragment may be detected by detecting a region of the fragment which comprises at least 4 amino acids including residue 34 of Aβ₁₋₄₀ (SEQ ID NO: 2). The at least one fragment may be detected by detecting a region of the fragment comprising residues 31-34 of Aβ₁₋₄₀ (SEQ ID NO: 2), preferably by detecting the C-terminal of SEQ ID NO: 6. In some embodiments, the at least one fragment is detected by detecting any one of the aforementioned regions comprising residue 34 or the C-terminal of SEQ ID NO: 6 and in addition detecting a region of the fragment comprising at least 4 amino acids including residue 21 of Aβ₁₋₄₀ (SEQ ID NO: 2), preferably a region of the fragment comprising residues 21-24 of Aβ₁₋₄₀ (SEQ ID NO: 2), and more preferably a region of the fragment comprising residues 21-29 of Aβ₁₋₄₀ (SEQ ID NO: 2). In some embodiments, the at least one fragment is detected by detecting the C-terminal of SEQ ID NO: 6 and detecting a region of the fragment comprising residues 21-24 of Aβ₁₋₄₀ (SEQ ID NO: 2), more preferably by detecting the C-terminal of SEQ ID NO: 6 and detecting a region of the fragment comprising residues 21-29 of Aβ₁₋₄₀ (SEQ ID NO: 2). In some embodiments, the at least one fragment is detected by detecting the C-terminal of SEQ ID NO: 6 and detecting SEQ ID NO: 7. In some embodiments, the at least one fragment is detected using an antibody which is specific to the C-terminal of SEQ ID NO: 6, preferably using an antibody which is specific for SEQ ID NO: 4. In some embodiments, the at least one fragment is detected using an antibody which is specific to the region of the fragment comprising at least 4 amino acids comprising residues 21-24 of Aβ₁₋₄₀ (SEQ ID NO: 2), preferably an antibody specific to SEQ ID NO: 7. In some embodiments, the at least one fragment is detected using an antibody specific to the C-terminal of SEQ ID NO: 6 and an antibody specific to SEQ ID NO: 7.

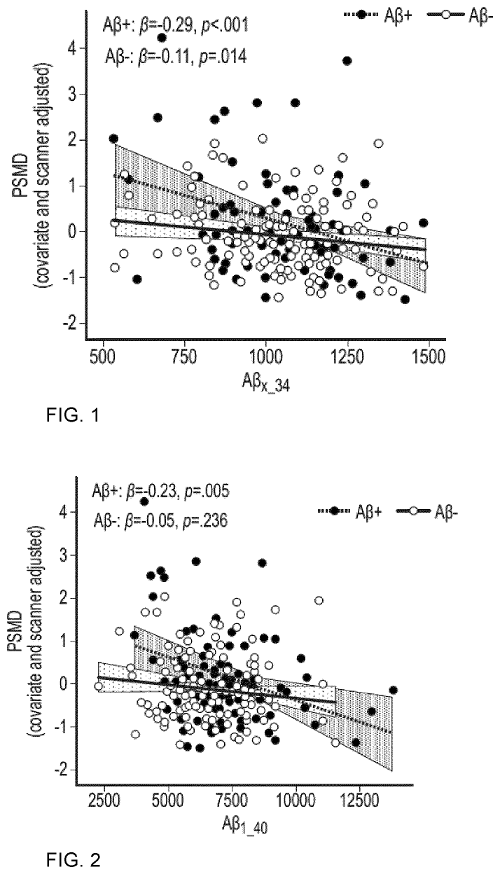

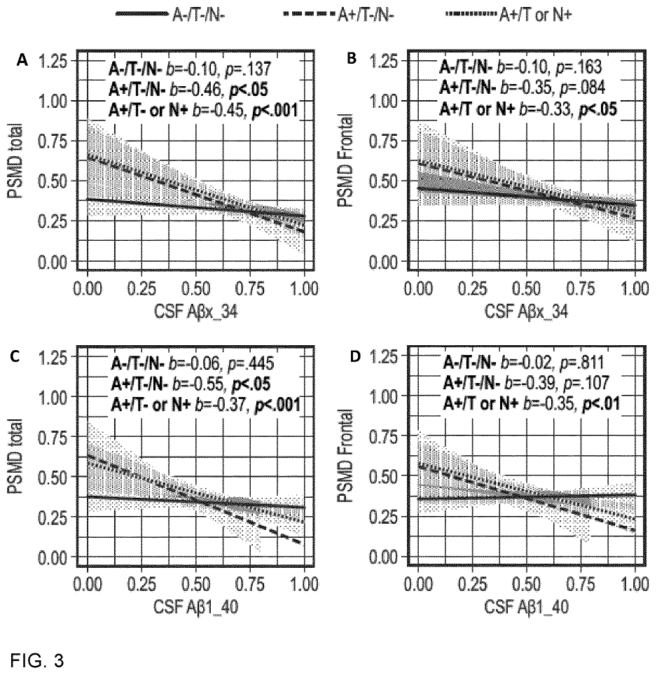

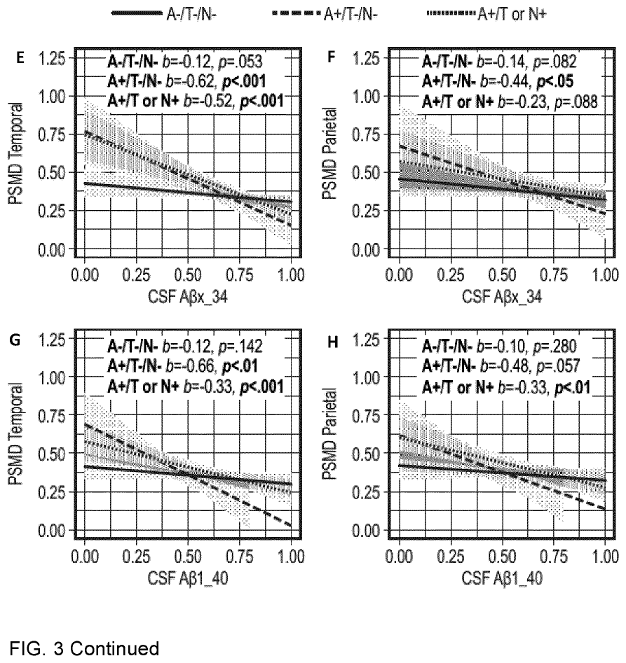

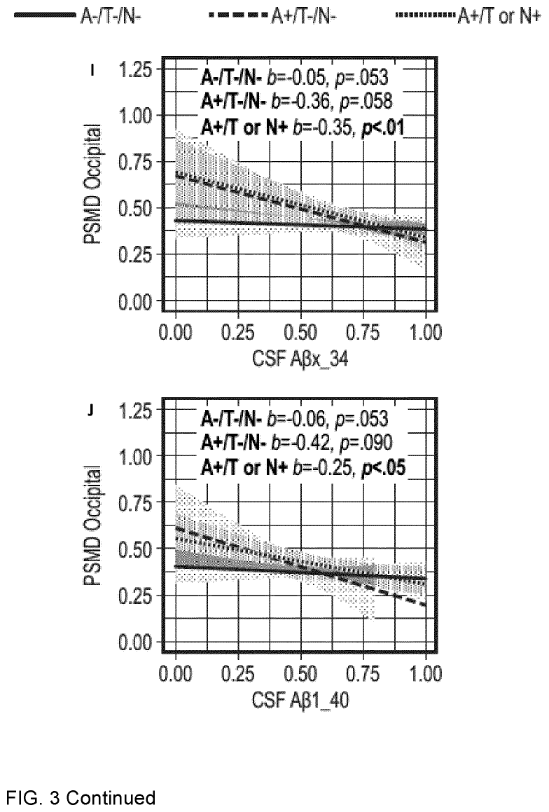

It is particularly preferable to determine the presence or quantity of Aβₓ₋₃₄ (i.e. the Aβₓ₋₃₄ fragment) because this measurement reflects the ongoing clearance of Aβ₁₋₄₀ by CNS pericytes. Defective pericytes catabolise less Aβ₁₋₄₀, thereby resulting in decreased amounts of Aβₓ₋₃₄. In addition, it has now been shown that Aβₓ₋₃₄ has a particularly robust relationship with PSMD in subjects with AD (i.e. positive markers for amyloid plaques; A+). In particular, FIGS. 1, 2 and 3 show that Aβₓ₋₃₄ has a more significant relationship to PSMD than Aβ₁₋₄₀, and FIG. 3 shows that is particularly the case for PSMD in the temporal lobe. FIG. 3 also shows that this significant relationship exists in subjects with early AD (i.e. A+/T−/N−) and more developed AD (A+/T or N+).

In some embodiments, the at least one fragment is Aβ₁₋₄₀ (SEQ ID NO: 2) which has been cleaved between amino acid residues 19 and 20, and between amino acid residues 34 and 35, such that the at least one fragment has the amino acid sequence shown in SEQ ID NO: 6. These fragments are referred to herein as “Aβ₂₀₋₃₄” or the “Aβ₂₀₋₃₄ fragment”. In these embodiments, the at least one fragment may be detected by detecting the N- or the C-terminal of SEQ ID NO: 6. In some embodiments, the at least one fragment may be detected by detecting the N- and the C-terminal of SEQ ID NO: 6. In some embodiments, the at least one fragment is detected using an antibody which is specific for the N- or the C-terminal of SEQ ID NO: 6, preferably an antibody which is specific for SEQ ID NO: 3 or SEQ ID NO: 4. In some embodiments, the at least one fragment may be detected using an antibody specific for the N-terminal of SEQ ID NO: 6 and an antibody which binds a fragment of Aβ₁₋₄₀ (SEQ ID NO: 2) comprising residue 34 of Aβ₁₋₄₀ (SEQ ID NO: 2) at its C-terminal. In some embodiments, the at least one fragment is detected using an antibody specific for the N-terminal of SEQ ID NO: 6 and using an antibody specific for the C-terminal of SEQ ID NO: 6. In some embodiments, the at least one fragment is detected using an antibody which is specific for SEQ ID NO: 3 and an antibody specific for SEQ ID NO: 4.

In some embodiments, the method comprises the step of detecting Aβ₁₋₄₀ (SEQ ID NO: 2), or at least one fragment of fragment thereof, by detecting the N-terminus of SEQ ID NO: 2. In some embodiments, the method comprises the step of detecting A₁₋₄₀ (SEQ ID NO: 2), or at least one fragment of fragment thereof, by detecting the C-terminus of SEQ ID NO: 2. These peptides and fragments thereof are referred to herein as “Aβₓ₋₄₀” or the “Aβₓ₋₄₀ fragment”. In these embodiments, the detection may be by detecting a region of the fragment comprising at least 4 amino acids including residue 40 of Aβ₁₋₄₀ (SEQ ID NO: 2). In some embodiments, the fragment or at least one fragment may be detected by detecting a region of at least 4 amino acids comprising residues 39-40 of Aβ₁₋₄₀ (SEQ ID NO: 2), preferably by detecting the C-terminal of SEQ ID NO: 2. In some embodiments, the fragment or at least one fragment may be detected by detecting a region of the fragment comprising residues 34-40 of Aβ₁₋₄₀ (SEQ ID NO: 2). In some embodiments, the fragment or at least one fragment may be detected by detecting any one of the aforementioned regions comprising residue 40 or the C-terminal of SEQ ID NO: 2 and, in addition, detecting a region of the fragment comprising at least 4 amino acids including residue 21 of Aβ₁₋₄₀ (SEQ ID NO: 2), preferably a region of the fragment comprising residues 21-24 of Aβ₁₋₄₀ (SEQ ID NO: 2), and more preferably a region of the fragment comprising residues 21-29 of Aβ₁₋₄₀ (SEQ ID NO: 2). In some embodiments, the fragment or at least one fragment is detected by detecting the C-terminal of SEQ ID NO: 2 and detecting a region of the fragment comprising residues 21-24 of Aβ₁₋₄₀ (SEQ ID NO: 2), more preferably the C-terminal of SEQ ID NO: 2 and a region of the fragment comprising residues 21-29 of Aβ₁₋₄₀ (SEQ ID NO: 2). In some embodiments, the fragment or at least one fragment may be detected by detecting a region of the fragment comprising residues 34-40 of Aβ₁₋₄₀ (SEQ ID NO: 2) and detecting a region of the fragment comprising residues 21-29 of Aβ₁₋₄₀ (SEQ ID NO: 2). In some embodiments, the fragment or at least one fragment may be detected using an antibody which is specific for any one of the above-mentioned regions or for the C-terminus of SEQ ID NO: 2. In some embodiments, the fragment or at least one fragment is detected using an antibody which is specific for SEQ ID NO: 5, an antibody which is specific for SEQ ID NO: 8 or an antibody which is specific for SEQ ID NO: 9. In some embodiments, the antibody specific SEQ ID NO: 9 is also specific for SEQ ID NO: 8 and/or SEQ ID NO: 5. In some embodiments, the antibody specific for SEQ ID NO: 8 is also specific for SEQ ID NO: 5, and vice versa. In some embodiments, the fragment or at least one fragment may be detected using an antibody which is specific for SEQ ID NO: 7 and an antibody specific for SEQ ID NO: 5, SEQ ID NO: 8 or SEQ ID NO: 9.

In some embodiments, the method comprises the step of detecting Aβ₂₀₋ₓ, Aβₓ₋₃₄, and Aβₓ₋₄₀. In other embodiments, the method comprises the step of detecting Aβ₂₀₋ₓ and Aβₓ₋₃₄; Aβ₂₀₋ₓ and Aβₓ₋₄₀; or Aβₓ₋₃₄ and Aβₓ₋₄₀. Each of these fragments can be detected using any of the methods described above. In some embodiments, each of these fragments is detected using antibodies specific for the N- or C-terminal of each, as described above.

In some embodiments, each of these fragments is detected using an antibody specific for SEQ ID NO: 3 or SEQ ID NO: 7, an antibody specific for SEQ ID NO: 4 and an antibody specific for SEQ ID NO: 5, SEQ ID NO: 8 or SEQ ID NO: 9. This is beneficial for assessing the efficacy of the MPS in clearing different fragments of Aβ.

In some embodiments, the method comprises determining the quantity of the Aβₓ₋₃₄ fragment and Aβ₁₋₄₀ (SEQ ID NO: 2) in the biological sample from the individual, and calculating the ratio the quantity of the Aβₓ₋₃₄ fragment to the quantity of Aβ₁₋₄₀ (SEQ ID NO: 2). The ratio of these quantities is indicative of the clearance (or defective clearance) of Aβ by the MPS, particularly pericytes. In particular, the sue of a ratio reduces variability in the method due to varying amounts of Aβ₁₋₄₀ between individuals. In some embodiments, a decrease in the ratio of the quantity of the Aβₓ₋₃₄ fragment to the quantity of Aβ₁₋₄₀ (i.e. a decrease in the quantity of Aβₓ₋₃₄ relative to the quantity of Aβ₁₋₄₀) is indicative of presence, predicted presence or development of the progress of amyloid angiopathy, ARIA or CAA in the individual. In some embodiments, the ratio is compared to the ratio of the quantity of the Aβₓ₋₃₄ fragment to Aβ₁₋₄₀ calculated from a biological sample previously obtained from the individual, to the ratio of the quantity of the Aβₓ₋₃₄ fragment to Aβ₁₋₄₀ calculated from a biological sample obtained from a healthy individual or to ratio of the quantity of the Aβₓ₋₃₄ fragment to Aβ₁₋₄₀ calculated from biological samples obtained from a plurality of healthy individuals, such that the decrease is with respect to any of these. Thus, it is possible to monitor the progress of amyloid angiopathy, CAA and ARIA by monitoring the change the ratio of the quantity of the Aβₓ₋₃₄ fragment to the quantity of Aβ₁₋₄₀, which is representative of changes in pericyte viability.

In some embodiments, the method comprises determining the quantity of the Aβₓ₋₃₄ fragment and the quantity of Aβ₁₋₄₀, and calculating the ratio thereof, in biological samples obtained from the individual at more than one time point, so as to obtain serial ratios over time. The ratio of the quantity of the Aβₓ₋₃₄ fragment to the quantity of Aβ₁₋₄₀ at each time point may be compared, such that a decrease in the ratio over time (i.e. a decrease in the quantity of Aβₓ₋₃₄ relative to the quantity of Aβ₁₋₄₀ over time) is indicative of the presence, predicted presence or development of the progress of amyloid angiopathy, ARIA or CAA in the individual. For example, the method may comprise determining the quantity of the Aβₓ₋₃₄ fragment and the quantity of A₁₋₄₀, and calculating the ratio thereof, in biological samples obtained from the individual at two, three, four or five time points. The time points may each be, for example, one, two, three, four, five or six months apart, or may each be one, two or three years apart. A decrease in the ratio of the quantity of the Aβₓ₋₃₄ fragment to the quantity of Aβ₁₋₄₀ across the time points is indicative of the presence, predicted presence, development of the progress of amyloid angiopathy, ARIA or CAA.

### Antibodies

In methods using antibodies to detect the Aβ protein fragment and/or at least one fragment thereof, a more generic antibody specific for Aβ₁₋₄₀ (SEQ ID NO: 2) or Aβₓ₋₃₄ may be used as part of the detection method. An exemplary antibody against Aβ₁₋₄₀ is sheep monoclonal AB.4D7 (Bioventix plc, UK) which can be used in an assay as a capture antibody in conjunction with a sheep monoclonal AB.2A9 (Bioventix plc, UK) for a mid-domain epitope (SEQ ID NO: 7) as the detection antibody. A Aβ₂₀₋ₓ assay may use the same AB.4D7 antibody as the capture antibody, and the highly specific 20-cut point sheep monoclonal antibody, ABNT.2B8 (Bioventix plc, UK) as the detection antibody. For a Aβₓ₋₃₄ assay, AB.2A9 may be used as the capture antibody and an anti-complex antibody, BVX.33420/H3 (Bioventix plc, UK), which preferentially binds a fragment having the C-terminal of SEQ ID NO: 6, may be used as the detection antibody, ensuring mid-domain x-34 specificity.

### Method of Treatment

In some embodiments, the method further comprises adjusting or tailoring the treatment of the individual based on the presence or quantity of the Aβ protein fragment in the biological sample. For example, if there is a decrease in the quantity of the Aβ protein fragment or a decrease in the ratio of the quantity of the Aβₓ₋₃₄ fragment to the quantity of Aβ₁₋₄₀ (i.e. a decrease in the quantity of Aβₓ₋₃₄ relative to the quantity of Aβ₁₋₄₀), then an anti-amyloid treatment may be not administered to the individual or the individual may be removed from an ongoing treatment (such as passive immunotherapy with an anti-amyloid drug), in order to prevent or reduce the risk of the development or progression or amyloid angiopathy, ARIA or CAA in the individual. As a further alternative, an anti-amyloid treatment to be administered to the individual may be selected depending on the presence or quantity of the Aβ protein fragment, the ratio of the quantity of the Aβₓ₋₃₄ fragment to the quantity of Aβ₁₋₄₀ or a decrease in either of these. In particular, as the method of the present invention allows for the detection, prediction or monitoring of the progress of development of amyloid angiopathy, ARIA or CAA, the method of the present invention can provide a method of personalised medicine which takes into account the presence, risk of or progression of amyloid angiopathy, CAA or ARIA. The method detects, predicts or monitors the progress of amyloid angiopathy, ARIA or CAA in the individual, in order to tailor the treatment of the individual.

In some embodiments, if there is a decrease in the quantity of the Aβ protein fragment or a decrease in the ratio of the quantity of the Aβₓ₋₃₄ fragment to the quantity of Aβ₁₋₄₀ (i.e. a decrease in the quantity of Aβₓ₋₃₄ relative to the quantity of Aβ₁₋₄₀), then the individual is removed from an ongoing treatment (such as passive immunotherapy with an anti-amyloid drug) and an anti-inflammatory treatment is administered to reduce inflammation associated with amyloid angiopathy, CAA or ARIA, and particularly ARIA.

### EXAMPLES

### Example 1

Amyloid angiopathy accompanies brain parenchymal amyloid deposits in AD. The established fluid biomarker Aβ₁₋₄₀ in the CSF or plasma is related to brain amyloid small vessel pathology, as measured by brain white matter imaging modalities. We exploited the Aβ cleavage pattern to develop antibodies targeting the meta-stable mid-domain N′- and C′-terminal epitopes at aa 20 and 34 in a novel assay, “Aβx-34”. This also relates to white matter imaging measures, and more clearly to a more vessel-specific measure like PSMD (“peak skeletonized mean diffusivity”) in amyloid-positive cases (A+), and thus is expected to provide more accurate information on incipient amyloid angiopathy. The results are shown in FIG. 1 (Aβₓ₋₃₄) and FIG. 2 (Aβ₁₋₄₀).

For the categorization of subjects as Aβ+ or Aβ−, the QuickPlex SQ 120 system from Meso Scale Discovery (MSD, MD, USA) was used to measure Aβ₁₋₄₂ and Aβ₁₋₄₀ in a multiplex setup using V-plex Ab Peptide Panel 1 (6E10) kit (K15200E-1). The CSF measurements of amyloid-β (Aβ)_{42/40} ratio was dichotomized to determine Aβ status as positive or negative. Subjects were stratified as A+ if CSF Aβ_{42/40} ratio ≤0.077, or A− if CSF Aβ_{42/40} ratio >0.077 ng/L, based on an amyloid-PET ¹⁸F-flutemetamol verified cut-off (Siafarikas et al., “Cerebrospinal fluid markers for synaptic function and Alzheimer type changes in late life depression”, Sci Rep 2021, 11, 20375). In subjects without CSF, amyloid-PET ¹⁸F-flutemetamol results alone were used to determine Aβ status. A subset of cases that were missing lumbar puncture or amyloid-PET ¹⁸F-flutemetamol at baseline were classified as Aβ+ if a lumbar puncture or amyloid-PET from a former assessment confirmed positive Aβ pathology, or Aβ− if more recent CSF or PET excluded Aβ pathology.

The immunoassay was a sandwich assay using a mid-domain antibody (2A9, Bioventix) bound to paramagnetic particles and a biotin-labelled anti-complex antibody (H3, Bioventix), which binds to the combined 2A9-Aβₓ₋₃₄ fragment. This configuration is highly specific to the cut point between residues 34 and 35 of Aβ, and detects all fragments starting between residues 1 and 22 of Aβ and ending with residue 34 of Aβ.

Aβ₁₋₄₀ was detected and measured using the Bioventix 3C11 antibody (epitope: positions 34-39) and the 3F3 antibody (epitope: positions 3-12).

In particular, FIGS. 1 and 2 show that there is a significant relationship between both Aβₓ₋₃₄ fragments and Aβ₁₋₄₀ fragments and PSMD in Aβ+ subjects. FIGS. 1 and 2 also show that there is a more robust relationship between CSF Aβₓ₋₃₄ and PSMD for both Aβ+ (established brain amyloid plaques) and Aβ− (no established brain amyloid plaques) subjects (FIG. 1: Aβ+, β=−0.29, β<0.001; Aβ−, β=−0.11, p=0.014) than between CSF Aβ₁₋₄₀ and PSMD, for both Aβ+ and Aβ− subjects (FIG. 2: Aβ+, P=−0.023, p=0.005; Aβ−, β=−0.05, p=0.236). Thus, it is expected that Aβₓ₋₃₄ fragments and Aβ₁₋₄₀ fragments, and particularly Aβₓ₋₃₄ fragments, can be used to detect and/or predict SVDs such as incipient amyloid angiopathy, CAA and ARIA.

### Example 2

### Background

Brain small vessel disease (SVD) increases Alzheimer's disease (AD) risk (Phlhaugen et al. “Brain amyloid and vascular risk are related to distinct white matter hyperintensity patterns”, J Cereb Blood Flow Metab, 2020, 41(5), p.1162-1174). Impaired amyloid β (Aβ) clearance is linked to amyloid plaque formation and cerebral amyloid angiopathy (CAA). Aβ₁₋₄₀ is deposited in vessel walls, and Aβ with a C-terminal 34′ cut-point is produced by perivascular cells. Evidence for SVD-AD interactions in early AD is elusive, but incipient CAA putatively increases susceptibility for amyloid-related imaging abnormalities (ARIA) in AD patients. “Peak width of skeletonized mean diffusivity” (PSMD) is a fully automated and robust imaging marker for SVD and can be easily applied to large samples (Baykara et al. “A novel imaging marker for Small Vessel Disease based on skeletonization of white matter tracts and diffusion histograms”, Ann Neurol, 2016 October, 80(4), p. 581-92; Horn et al. “Peak width of skeletonized mean diffusivity and cognitive performance in cerebral amyloid angiopathy”, Front. Neurosci., 2023, vol. 17). Here, the SVD MRI marker PSMD was compared to cerebrospinal fluid (CSF) Aβ species, assessing links between SVD and AD pathologies and Aβ species as potential markers for incipient CAA.

### Methods

Cross-sectional data from the Dementia Disease Initiation (DDI) cohort were available, comprising (n=112) cognitively normal (CN) and (n=115) cases with mild cognitive impairment (MCI) (total n=227).

The subjects were classified according to the A/T/N system, which is an unbiased description based on care biomarkers of AD, defined by presence (+) or absence (−) of amyloid pathology (A), tangle pathology (T) or neurodegeneration (N) (Jack et al., “A/T/N: An unbiased descriptive classification scheme for Alzheimer disease biomarkers”, Neurology 2016, 87, 539-547), here biomarkers based on cerebrospinal fluid (CSF) biomarkers as described in Jack et al. The CSF measurements of amyloid-β (Aβ)_{42/40} ratio, phosphorylated tau (p-tau) and total tau (t-tau) were dichotomized to determine A, T and N status as positive or negative. A/T/N staging was performed as follows: A−/T−/N− (biomarker negative controls), A−/T+ or N+, A+/T−/N−, and A+/T+ or N+. According to A/T/N-classification, A−T−/N− was defined as controls, and A−/T+ or N+, A+/T−/N−, and A+/T+ or N+ were defined as cases.

Subjects were stratified as A+ if CSF Aβ_{42/40} ratio ≤0.077, or A− if CSF Aβ_{42/40} ratio >0.077 ng/L, based on an amyloid-PET ¹⁸F-flutemetamol verified cut-off (Siafarikas et al., “Cerebrospinal fluid markers for synaptic function and Alzheimer type changes in late life depression”, Sci Rep 2021, 11, 20375). In subjects without CSF, amyloid-PET ¹⁸F-flutemetamol results alone were used to determine Aβ status. A subset of cases that were missing lumbar puncture or amyloid-PET ¹⁸F-flutemetamol at baseline were classified as Aβ+ if a lumbar puncture or amyloid-PET from a former assessment confirmed positive Aβ pathology, or Aβ− if more recent CSF or PET excluded Aβ pathology. The same approach was used to determine total or phosphorylated tau when CSF total or phosphorylated tau were missing at baseline.

Cerebrospinal fluid (CSF) markers for AD A/T/N-staging identified 134 cases with negative markers (A−/T−/N−; i.e. subjects without AD), 21 with amyloid positive markers (A+/T−/N−; representative of subjects with early AD) and 72 with amyloid and tau-positive markers (A+/T or N+; representative of subjects with more advanced AD than A+/T−/N− subjects).

For the categorization of subjects for A/T/N grading, commercial enzyme-linked immunosorbent assays (Innotest, Fujirebio, Ghent, Belgium) based on monoclonal antibodies were used to measure CSF concentrations of total tau (t-tau) using hTau Ag kits, and phosphorylated tau (p-tau) using 181P kits. The QuickPlex SQ 120 system from Meso Scale Discovery (MSD, MD, USA) was used to measure Aβ₁₋₄₂ and Aβ₁₋₄₀ in a multiplex setup using V-plex Ab Peptide Panel 1 (6E10) kit (K15200E-1). The analyses were carried out according to the manufacturers' procedures.

The Aβₓ₋₃₄ immunoassay was a sandwich assay using a mid-domain antibody (2A9, Bioventix) bound to paramagnetic particles and a biotin-labelled anti-complex antibody (H3, Bioventix), which binds to the combined 2A9-Aβₓ₋₃₄ fragment. This configuration is highly specific to the cut point between residues 34 and 35 of Aβ, and detects all fragments starting between residues 1 and 22 of Aβ and ending with residue 34 of Aβ.

For the comparison to PSMD, Aβ₁₋₄₀ was detected and measured using the Bioventix 3C11 antibody (epitope: positions 34-39) and the 3F3 antibody (epitope: positions 3-12).

Table 1 sets out the sample characteristics. Multiple linear regression models were used with PSMD by brain lobe as the dependent variable, and NfL, Aβ₁₋₄₀ (MesoScale), Aβₓ₋₃₄ (Bioventix, Pre Diagnostic) (separately) and interactions with A/T/N group as independent variables. Variables were log-transformed and standardised.

TABLE 1
Between-group comparisons of sample characteristics and CSF Aβₓ₋₃₄, Aβ₁₋₄₀ and NfL.

| | A/T/N groups (n) | | | | Statistical tests | | |
|---|---|---|---|---|---|---|---|
| | Cases | Cases | Cases | | A−/N− | A−/N− | A+/N− |
| | A−/T−/N− | A+/T−/N− | A+/T or N+ | F/χ²/H/η²/ | vs | vs | vs |
| | (134) | (21) | (72) | ηₚ² (p) | A+/N− | A+/N+ | A+/N+ |
| Age | 59.9 | 69.0 | 69.4 | F = 32.5, | ^{c}<.001 | ^{c}<.001 | ^{c}n.s. |
| Mean | (9.3) | (69.9) | (7.9) | η² = .21, | | | |
| (SD) | | | | (<.001) | | | |
| Female | 79 | 11 | 34 | χ² = 2.6, | ^{a} | ^{a} | ^{a} |
| n (%) | (59) | (52) | (47) | (n.s.) | | | |
| MMSE | 29 | 29 | 28 | H = 28.5, | ^{e}n.s. | ^{e}<.001 | ^{e}n.s. |
| Median (IQR) | (2) | (3) | (1) | (<.001) | | | |
| CSF Aβx_34 | 1047.1 | 982.3 | 1124.9 | F = 7.0 | ^{d}n.s. | ^{d}<.01 | ^{d}<.01 |
| Mean (SD) | (197.7) | (200.2) | (184.6) | ηₚ² = .06, | | | |
| | | | | (<.01) | | | |
| CSF Aβ1_40 | 6515.7 | 6117.8 | 7879.8 | F = 18.6 | ^{d}n.s. | ^{d}<.001 | ^{d}<.001 |
| Mean (SD) | (1307.8) | (1145.7) | (1938.0) | ηₚ² = .14, | | | |
| | | | | (<.001) | | | |
| CSF NfL | 2704.4 | 3307.8 | 4323.6 | F = 8.8 | ^{d}n.s. | ^{d}<.001 | ^{d}<.05 |
| Mean (SD) | (1498.5) | (1032.2) | (1691.8) | ηₚ² = .07, | | | |
| | | | | (<.001) | | | |
| CN n % | 83 | 8 | 21 | ^{b} | ^{a} | ^{a} | ^{a} |
| | (61.9) | (38.1) | (29.2) | | | | |
| MCI n % | 51 | 13 | 51 | ^{b} | ^{a} | ^{a} | ^{a} |
| | (38.1) | (69.1) | (70.8) | | | | |

Abbreviations:
A+/−, positive or negative CSF marker for amyloid plaques;
T+/−, positive or negative marker for tau-tangles (p-tau);
N+/−, positive or negative marker for neurodegeneration (t-tau);
SD, standard deviation;
n, number of cases;
%, percentage;
F, F statistic;
χ², chi square statistic;
H, Kruskal-wallis statistic;
η², eta-squared;
ηₚ², partial eta-squared;
vs, versus;
^{a}, no post-hoc comparisons performed;
^{b}, no value;
^{c}ANOVA post-hoc comparisons (unadjusted p-values);
^{d}ANCOVA post-hoc comparisons adjusted for age and sex (unadjusted p-values);
^{e}Dunn's pairwise post-hoc comparisons (unadjusted p-values).

### Results

In A+ (i.e. subjects positive for amyloid plaques), lower Aβ markers were related to higher PSMD in all lobes, but particularly for the temporal lobe (FIG. 3). Aβₓ₋₃₄ showed more robust relationships with PSMD in both A+ groups particularly in the temporal lobes (A+/T−/N−: b=0.62, p=<0.001; A+/T or N+: b=0.52, p<0.001) than Aβ₁₋₄₀ (A+/T−/N−: b=0.66, p=<0.01; A+/T or N+: b=0.33, p<0.001).

### Discussion

CSF Aβₓ₋₃₄ was particularly tightly linked to increased temporal lobe PSMD, even in A+/T−/N− (i.e. early AD), pointing to early AD-related SVD. This supports that CSF Aβₓ₋₃₄ can be used as a marker for CAA and as a susceptibility marker for ARIA.

Sequences

| SEQ ID NO: | Sequence |
|---|---|
| 1 | Asp Ala Glu Phe Arg His Asp Ser Gly Tyr Glu Val His His |
| | Gln Lys Leu Val Phe Phe Ala Glu Asp Val Gly Ser Asn Lys |
| | Gly Ala Ile Ile Gly Leu Met Val Gly Gly Val Val Ile Ala |
| 2 | Asp Ala Glu Phe Arg His Asp Ser Gly Tyr Glu Val His His |
| | Gln Lys Leu Val Phe Phe Ala Glu Asp Val Gly Ser Asn Lys |
| | Gly Ala Ile Ile Gly Leu Met Val Gly Gly Val Val |
| 3 | Phe Ala Glu Asp Val Gly |
| 4 | Gly Ala Ile Ile Gly Leu |
| 5 | Val Val Ile Ala |
| 6 | Phe Ala Glu Asp Val Gly Ser Asn Lys Gly Ala Ile Ile Gly |
| | Leu |
| 7 | Ala Glu Asp Val Gly Ser Asn Lys Gly |
| 8 | Leu Met Val Gly Gly Val Val Ile |
| 9 | Leu Met Val Gly Gly Val Val |

1-13. (canceled)

14. A method of detecting an Aβ protein fragment in a sample, comprising:
detecting the presence or quantity of an Aβ protein fragment in a biological sample obtained from an individual having or suspected of having amyloid angiopathy, amyloid-related imaging abnormalities (ARIA), or cerebral amyloid angiopathy (CAA), wherein the Aβ protein fragment is an Aβₓ₋₃₄ fragment or is a fragment of Aβ₁₋₄₀.

15. A method of detecting an Aβ protein fragment, comprising (i) obtaining a biological sample from an individual having or suspected of having amyloid angiopathy, amyloid-related imaging abnormalities (ARIA), or cerebral amyloid angiopathy (CAA) and (ii) detecting the Aβ protein fragment in the biological sample, wherein the Aβ protein fragment is an Aβₓ₋₃₄ fragment or a fragment of Aβ₁₋₄₀.

16. The method of claim 15, wherein the biological sample is a CSF sample or a blood sample.

17. The method of claim 15, wherein the fragment of Aβ₁₋₄₀ has the amino acid sequence of SEQ ID NO: 2 and the Aβₓ₋₃₄ fragment consists of a fragment of SEQ ID NO:2 having at its N-terminal an amino acid at a position of amino acid residue 21 or lower of SEQ ID NO:2 and at its C-terminal the amino acid at amino acid residue 34 of SEQ ID NO:2.

18. The method claim 15, wherein detecting the Aβₓ₋₃₄ fragment or the fragment of Aβ₁₋₄₀ comprises detecting a region of Aβ₁₋₄₀ comprising at least 4 amino acids including residue 21, residue 34, or residue 40 of Aβ₁₋₄₀ (SEQ ID NO: 2).

19. The method of claim 15, wherein detecting the Aβₓ₋₃₄ fragment or the fragment of Aβ₁₋₄₀ comprises detecting a region of the fragment comprising at least 4 amino acids comprising residues 21-24, residues 31-34, or residues 39-40 of Aβ₁₋₄₀ (SEQ ID NO: 2).

20. The method of claim 15, wherein detecting the Aβₓ₋₃₄ fragment or the fragment of Aβ₁₋₄₀ comprises detecting one or more of the N-terminal of SEQ ID NO: 6, the C-terminal of SEQ ID NO: 6, a region of the fragment comprising residues 21-29 of Aβ₁₋₄₀ (SEQ ID NO: 2), and a region of the fragment comprising residues 34-40 of Aβ₁₋₄₀ (SEQ ID NO: 2).

21. The method of claim 20, wherein detecting the Aβₓ₋₃₄ fragment or the fragment of Aβ₁₋₄₀ comprises detecting more than one of the N-terminal of SEQ ID NO: 6, the C-terminal of SEQ ID NO: 6, and the regions of Aβ₁₋₄₀.

22. The method of claim 15, wherein the fragment of Aβ₁₋₄₀ is a fragment which has cleaved between amino acid residues 19 and 20 and/or between amino acid residues 34 and 35.

23. The method of claim 15, wherein detecting the Aβₓ₋₃₄ fragment or the fragment of Aβ₁₋₄₀ comprises using antibodies to detect the Aβₓ₋₃₄ fragment or the fragment of Aβ₁₋₄₀ and an antibody specific for the C-terminal of SEQ ID NO: 6 or specific for SEQ ID NO: 4.

24. The method of claim 15, wherein the method comprises detecting the quantity of the Aβₓ₋₃₄ fragment in the biological sample and the quantity of Aβ₁₋₄₀ (SEQ ID NO: 2) in the biological sample, and calculating a ratio thereof.

25. The method of claim 24, wherein a decrease in the quantity of the Aβₓ₋₃₄ fragment relative to the quantity of Aβ₁₋₄₀ is indicative of the presence, predicted presence, or development of the progress of amyloid angiopathy, ARIA, or CAA in the individual.

26. The method of claim 25, wherein the decrease is with respect to the ratio of the quantity of the Aβₓ₋₃₄ fragment to Aβ₁₋₄₀ calculated from a biological sample previously obtained from the individual, or with respect to the ratio of the quantity of the Aβₓ₋₃₄ fragment to Aβ₁₋₄₀ calculated from a biological sample obtained from a healthy individual, or with respect to the ratio of the quantity of the Aβₓ₋₃₄ fragment to Aβ₁₋₄₀ calculated from biological samples obtained from a plurality of healthy individuals.

27. The method of claim 24, wherein the method comprises detecting the quantity of the Aβₓ₋₃₄ fragment and the quantity of Aβ₁₋₄₀ (SEQ ID NO: 2) in biological samples obtained from the subject at more than one time point, and calculating a ratio thereof for each time point, so as to obtain serial ratios over time.

28. The method of claim 17, wherein the Aβₓ₋₃₄ fragment comprises or consists of the amino acid sequence of SEQ ID NO: 6.

29. The method of claim 21, wherein detecting the Aβₓ₋₃₄ fragment or the fragment of Aβ₁₋₄₀ comprises detecting:
(i) a region of the fragment comprising at least 4 amino acids comprising residues 21-24 of Aβ₁₋₄₀ and a region of the fragment comprising at least 4 amino acids comprising residues 31-34 or 39-40 of Aβ₁₋₄₀;
(ii) the N-terminal of SEQ ID NO: 6 and a region of the fragment comprising residues 34-40 of or Aβ₁₋₄₀ of Aβ₁₋₄₀;
(iii) the C-terminal of SEQ ID NO: 6 and a region of the fragment comprising residues 21-24 of or Aβ₁₋₄₀; and/or
(iv) a region of the fragment comprising residues 21-24 of Aβ₁₋₄₀ and a region of the fragment comprising residues 34-40 of Aβ₁₋₄₀.

30. The method of claim 29, wherein the detection of the N-terminal of SEQ ID NO: 6 and a region of the fragment comprising residues 34-40 of or Aβ₁₋₄₀ comprises the detection of residues 1-6 of SEQ ID NO: 6 and a region of the fragment comprising residues 34-40 of Aβ₁₋₄₀.

31. The method of claim 23, wherein detecting the Aβₓ₋₃₄ fragment or the fragment of Aβ₁₋₄₀ comprises using an antibody specific for SEQ ID NO: 3 or SEQ ID NO: 7.

32. The method of claim 23, wherein detecting the Aβₓ₋₃₄ fragment or the fragment of Aβ₁₋₄₀ comprises also using an antibody specific for SEQ ID NO: 5, SEQ ID NO: 8, or SEQ ID NO: 9.


SEQUENCE LISTING (9 sequences)

SEQ ID NO: 1 - protein, 42 aa, Homo sapiens [sequence omitted]

SEQ ID NO: 2 - protein, 40 aa, Homo sapiens
DAEFRHDSGYEVHHQKLVFFAEDVGSNKGAIIGLMVGGVV

SEQ ID NO: 3 - protein, 6 aa, Homo sapiens
FAEDVG

SEQ ID NO: 4 - protein, 6 aa, Homo sapiens
GAIIGL

SEQ ID NO: 5 - protein, 4 aa, Homo sapiens
VVIA

SEQ ID NO: 6 - protein, 15 aa, Homo sapiens
FAEDVGSNKGAIIGL

SEQ ID NO: 7 - protein, 9 aa, Homo sapiens
AEDVGSNKG

SEQ ID NO: 8 - protein, 8 aa, Homo sapiens
LMVGGVVI

SEQ ID NO: 9 - protein, 7 aa, Homo sapiens
LMVGGVV